(19) United States
(12) Patent Application Publication
(10) Pub. No.: US 2012/0121544 A1
(43) Pub. Date: May 17, 2012

(54) Method for Efficiently Proliferating and Differentiating Natural Killer Cells from Umbilical Cord Blood

(75) Inventors: Inpyo Choi, Daejeon (KR); Suk-Ran Yoon, Daejeon (KR); Soo Yun Lee, Daejeon (KR); Joo-Yong Lee, Daejeon (KR); Jaeseung Lim, Kyounggi-do (KR)

(73) Assignee: MEDICELL CORPORATION, Daejeon (KR)

(21) Appl. No.: 13/125,643

(22) PCT Filed: Sep. 28, 2009
(86) PCT No.: PCT/KR2009/005531

§ 371 Date: Jun. 3, 2011

(30) Foreign Application Priority Data
Oct. 24, 2008 (KR) 10-2008-0104774

(51) Int. Cl. A61K 35/12; A61P 35/00; C12N 5/02

(52) U.S. Cl. 424/93.7; 435/375; 435/377

(57) ABSTRACT

The present invention relates to a method for efficiently proliferating and differentiating natural killer cells (NK cells) from umbilical cord blood, more precisely a method for efficiently proliferating and differentiating natural killer cells from umbilical cord blood comprising the following steps: 1) preparing CD3 negative cells by eliminating CD3 positive T-cells from umbilical cord blood derived mononuclear cells; and 2) culturing the CD3 negative cells after treating various cytokines thereto. The present invention is advantageous in obtaining high purity NK cells in a short period of time by inducing NK cells from CD3 negative cells, compared with the conventional method inducing NK cells from haematopoietic stem cells, and in promoting NK cell proliferation and differentiation by treating different cytokines together, for example by the co-treatment of IL-15 and IL-21. That is, the method of the present invention can induce NK cells with increased anti-cancer cytotoxicity, so that it can be effectively used for anti-cancer cell therapy.

### BACKGROUND OF THE INVENTION

1. Field of the Invention

The present invention relates to a method for efficiently proliferating and differentiating natural killer cells from umbilical cord blood, more precisely a method for efficiently proliferating and differentiating natural killer cells from umbilical cord blood comprising the following steps: 1) preparing CD3 negative cells by eliminating CD3 positive T-cells from umbilical cord blood derived mononuclear cells; and 2) culturing the CD3 negative cells after treating various cytokines thereto.

2. Description of the Related Art

Among the cells forming immune system, natural killer cells (referred, as “NK cells” hereinafter) are known to be able to kill cancer cells non-specifically. This anti-tumor cytotoxicity of NK cells has been utilized to treat solid tumor by using lymphokine activated killer cells (LAK) and tumor infiltration lymphocytes (TIL), or has been applied for the new attempt of cell therapy to avoid rejection response occurring after bone marrow transplantation or organ transplantation via immune therapy based on donor lymphocyte infusion (Tilden. A. B. et al., J. Immunol., 136: 3910-3915, 1986; Bordignon C, et al., Hematologia 84: 1110-1149, 1999). It has been also reported that the defect of NK cell differentiation and activation is closely related to different kinds of cancers such as breast cancer (Konjevic G, et al., Breast Cancer Res. Treat., 66: 255-263, 2001), melanoma (Ryuke Y, et al., Melanoma Res., 13: 349-356, 2003), and lung cancer (Villegas F R, et al., Lung Cancer, 35: 23-28, 2002), etc. So, the novel approach of cell therapy using NK cells attracts our attention in order to treat such diseases.

B cells and T cells are still found in a cytokine receptor γ_{c} deficient mouse, but NK cells are not. Therefore, it is assumed that those receptors having γ_{c} play an important role in NK cell differentiation (Singer, B et al., Proc. Natl. Acad. Sci. USA 92, 377-381, 1995). Types of receptor γ_{c} are IL-2, IL-4, IL-7, IL-9, IL-15, and IL-21, among which IL-2 is reported to be functioning to increase the proliferation and activation of mature NK cells (Shibuya, A. et al., Blood 85, 3538-3546, 1995). IL-2 deficient human and mouse showed remarkably decreased NK cell population (DiSanto, J. P. et al., J. Exp. Med. 171, 1697-1704, 1990). In the meantime, studies proved that IL-2 and IL-2Ra deficiency affects the activity and the population of NK cells indirectly. It is also known that IL-2R chain is involved in the formation of IL-15 receptor.

IL-15 is involved in NK cell differentiation. NK cell deficiency is observed in the mouse lacking of transcription factor interferon (IFN)-regulation factor 1 required for the generation of IL-15 (Kouetsu et al., Nature 391, 700-703, 1998), which has been supported by the study showing that NK cells are not found in an IL-15 or IL-15Ra deficient mouse. It has been reported that IL-15 enhances directly the growth and the differentiation of NK cells via IL-15 receptor expressed in NK cells (MrozekE et al., Blood 87, 2632-2640, 1996).

IL-21 is the cytokine secreted by activated CD4⁺T cells (Nature, 5:688-697, 2005). IL-21R is expressed in dendritic cells, NK cells, and lymphocytes such as T-cells and B-cells (Rayna Takaki, et al., J. Immonol 175: 2167-2173, 2005). The structure of IL-21 is very similar to those of IL-2 and IL-15. IL-21R shares its chain with IL-2R, IL-15, IL-7R, and IL-4R, etc. (Asao et al., J. Immunol, 167: 1-5, 2001). According to the previous report, IL-21 induces the maturation of NK cell precursor from bone marrow (Parrish-Novak, et al., Nature, 408: 57-63, 2000). In particular, IL-21 has been reported to increase effector functions of NK cells such as cytokine generation and cytotoxicity (M. Strengell, et al., J Immunol, 170: 5464-5469, 2003; J. Brady, et al., J Immunol, 172: 2048-2058, 2004). It also increases effector functions of CD8⁺T cells, resulting in the promotion of anti-cancer response of adaptive immune system (Rayna Takaki, et al., J Immunol 175: 2167-2173, 2005; A. Moroz, et al., J Immunol, 173: 900-909, 2004). Moreover, it activates NK cells separated from human peripheral blood (Parrish-Novak, et al., Nature, 408: 57, 2000), and plays an important role in inducing maturation of NK cells from hematopoietic stem cells separated from umbilical cord blood (J. Brady, et al., J Immunol, 172: 2048, 2004).

To utilize NK cells efficiently for anti-cancer immuno cell therapy, it is important to secure enough numbers of NK cells. However, NK cells only take 10-15% of lymphocytes in blood, and the population, differentiation, and function of NK cells are all reduced in a cancer patient. Therefore, it is actually very hard to secure enough numbers of NK cells. Thus, it is urgently required to develop a method to secure a huge number of NK cells via NK cell proliferation or differentiation.

NK cells are originated from hematopoietic stem cells (HSC) in bone marrow. Previous methods of inducing differentiation of NK cells in vitro are based on the culture of hematopoietic stem cells separated from umbilical cord blood and pre-treated with appropriate cytokines (Galy et al., Immunity 3: 459-473, 1995; Mrozek E, et al., Blood 87:2632-2640, 1996; Sivori, S. et al., Eur J. Immunol. 33:3439-3447, 2003; B. Grzywacz, et al., Blood 108: 3824-3833, 2006). That is, Flt-3L, IL-7, SCF, and IL-15 are added to CD34⁺ HSC, followed by culture for 5 weeks to induce the differentiation into CD3⁻CD56⁺ NK cells. However, it is hard to obtain enough numbers of cells for the treatment and it takes long time and high costs to induce that differentiation, which makes its clinical use difficult.

It has been known up to this day that NK cells are originated from CD34⁺ HSC. But, they are differentiated via multiple precursor steps. The most representative precursor is CD122⁺ cell, even though all the precursors have not been identified, yet. There have been no reports saying that CD3⁻ cells are the NK precursors.

The present inventors tried to develop a more efficient and economical method for obtaining NK cells. As a result, the present inventors completed this invention by confirming that the novel method consisting of the steps of preparing CD3 negative cells by eliminating CD3 positive cells from mononuclear cells isolated from umbilical cord blood and culturing the CD3 negative cells which have been pre-treated with cytokines such as IL-15 and IL-21 could promote NK cell growth and differentiation efficiently and increase cytotoxicity of NK cells.

### SUMMARY OF THE INVENTION

It is an object of the present invention to provide a method for efficiently proliferating and differentiating NK cells from umbilical cord blood which consists of the steps of 1) preparing CD3 negative cells by eliminating CD3 positive T cells from umbilical cord blood derived mononuclear cells; and 2) culturing the CD3 negative cells after pre-treating the cells with various cytokines and thereby the conditions for the proliferation and differentiation of NK cells are optimized.

It is another object of the present invention to provide a preventive and therapeutic composition for cancer that contains NK cells with increased cytotoxicity which is prepared by the said method of the present invention, and to provide a preventive and therapeutic method for cancer using the NK cells with increased cytotoxicity.

To achieve the above objects, the present invention provides a method for proliferating NK cells comprising the following steps:

1) preparing CD3 negative cells by eliminating CD3 positive T cells from umbilical cord blood derived mononuclear cells; and

2) culturing the CD3 negative cells after treating the cells of step 1) with cytokines.

The present invention also provides a method for differentiating NK cells comprising the following steps:

1) preparing CD3 negative cells by eliminating CD3 positive T cells from umbilical cord blood derived mononuclear cells; and

2) culturing the CD3 negative cells after treating the cells of step 1) with cytokines.

The present invention further provides a method for preparing NK cells with increased cytotoxicity comprising the following steps:

1) preparing CD3 negative cells by eliminating CD3 positive T cells from umbilical cord blood derived mononuclear cells; and

2) culturing the CD3 negative cells after treating the cells of step 1) with cytokines.

The present invention also provides a preventive and therapeutic composition for cancer containing the NK cells with increased cytotoxicity prepared by the said method.

The present invention also provides a method for treating cancer containing the step of administering a pharmaceutically effective dosage of the said composition to a subject having cancer.

The present invention also provides a method for preventing cancer containing the step of administering a pharmaceutically effective dosage of the said composition to a subject.

In addition, the present invention provides a use of the NK cells with increased cytotoxicity prepared by the method of the present invention for the production of a preventive and therapeutic composition for cancer.

### ADVANTAGEOUS EFFECT

The method for proliferating and differentiating NK cells of the present invention can produce NK cells with higher purity compared with the method using the conventional hematopoietic stem cells by inducing NK cell differentiation from CD3 negative cells, and favors for the proliferation and differentiation of NK cells by co-treating the cells with different cytokines, IL-15 and IL-21. As a result, the method can produce NK cells with increased cytotoxicity, which thus can be effectively used for the anti-cancer treatment.

### BRIEF DESCRIPTION OF THE DRAWINGS

The application of the preferred embodiments of the present invention is best understood with reference to the accompanying drawings, wherein:

FIG. 1 is a set of graphs illustrating the results of two-color flow cytometry. Mononuclear cells (MNC) were isolated from human umbilical cord blood. Then, CD3 negative cells were obtained by eliminating CD3 positive T-cells therefrom. Two-color flow cytometry was performed to investigate the purity of CD3 negative cells and the mononuclear cells.

FIG. 2 is a graph illustrating the changes of CD3 negative cell population over the time after treating CD3 negative cells with cytokines (IL-2, IL-15, and IL-21), which were observed to investigate the effect of cytokines on CD3 negative cell growth.

FIG. 3 is a set of graphs illustrating the NK cell (CD3⁻CD56⁺) ratio analyzed by FACS to investigate the effect of cytokines (IL-2, IL-15, and IL-21) on NK cell differentiation. CD3 negative cells were treated with different combinations of the said cytokines, followed by culture. Then, FACS was performed to analyze NK cell ratio.

FIG. 4 is a graph illustrating the statistics analyzing NK cell (CD3⁻CD56⁺) ratio, which was performed to investigate the direct effect of cytokines (IL-2, IL-15, and IL-21) on NK cell differentiation. CD3 negative cells were treated with different combinations of cytokines, followed by culture. Then, NK cell ratio was investigated.

FIG. 5 is a set of graphs illustrating the results of FACS showing cultured NK cell ratios in the groups treated with two combinations of cytokines (IL-15+IL-21, IL-2+IL-15+IL-21), which would be believed to be the most effective combinations in inducing differentiation and proliferation.

FIG. 6 is a graph illustrating the result of ⁵¹Cr secretion assay with NK cell groups cultured after treated with the two most effective combinations of cytokines (IL-15+IL-21, IL-2+IL-15+IL-21) in inducing differentiation and proliferation, which was performed to investigate the direct effect of cytokines on NK cell cytotoxicity.

FIG. 7 is a graph illustrating the result of LDH activity assay with NK cell groups cultured after treated with the two most effective combinations of cytokines (IL-15+IL-21, IL-2+IL-15+IL-21) in inducing differentiation and proliferation, which was performed to investigate the direct effect of cytokines on NK cell cytotoxicity.

### DESCRIPTION OF THE PREFERRED EMBODIMENTS

The terms used in this invention are described hereinafter.

The term “preventing” used in this invention indicates every action that can suppress the outbreak or the metastasis of cancer or delay the progress of cancer by administering the composition of the present invention.

The terms “treating” and “improving” used in this invention indicate every action that can change the symptoms of cancer or metastasis advantageously by administering the said composition of the present invention.

The term “administration” used in this invention indicates the action to provide a required amount of the composition of the present invention to a subject according to an appropriate method.

The term “subject” used in this invention indicates human or any animal including monkey, dog, goat, pig, and rat, in which cancer development or metastasis might be suppressed by the administration of the composition of the present invention.

The term “pharmaceutically effective dosage” used in this invention indicates the amount of the composition that is reasonably accepted for the clinical treatment or enough amounts to suppress cancer development or metastasis. This dosage can be determined according to kinds of disease, severity of disease, pharmaceutical activity, sensitivity, administration term and pathway, excretion rate, period of treatment, and other drugs which would be used together, as well as other factors well-known in the field of medicine.

Hereinafter, the present invention is described in detail.

The present invention provides a method for proliferating NK cells comprising the following steps:

1) preparing CD3 negative cells by eliminating CD3 positive T cells from umbilical cord blood derived mononuclear cells; and

2) culturing the CD3 negative cells after treating the cells of step 1) with cytokines.

In the above method, the preparation of CD3 negative cells in step 1) can be accomplished by either the method of using CD3 microbeads to give CD positive cells magnetism which would be filtered by MACS column to separate CD3 negative cells therefrom or the method of labeling CD3 negative T cells with fluorescence which would be separated by using Cell Sorter, but not always limited thereto.

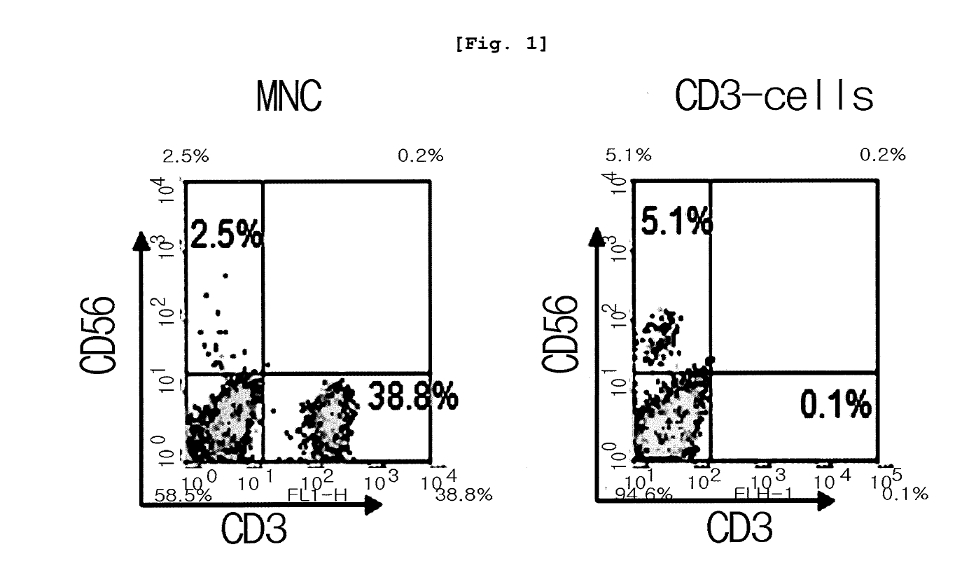

To prepare CD3 negative cells in this invention, mononuclear cell layer (MNC layer) was first isolated from umbilical cord blood. Then, mononuclear cells were obtained by eliminating erythrocytes from the MNC layer. CD3 microbeads (Miltenyi Biotech) were added thereto to give magnetism to CD3⁺ cells, which were passed through MACS column to separate CD3⁻ cells and CD3⁺ cells. As a result, CD3⁻ cells were recovered from mononuclear cells (yield: 32%). The purity of CD3 negative cells was 88% at average (see Table 1, Table 2, and FIG. 1).

In the above method, cytokines in step 2) are preferably at least 2 selected from the group consisting of IL2, IL-15, and IL-21, and it is more preferred to co-treat IL-15 and IL-21 together or Il-2, IL-15, and IL21 altogether, but not always limited thereto.

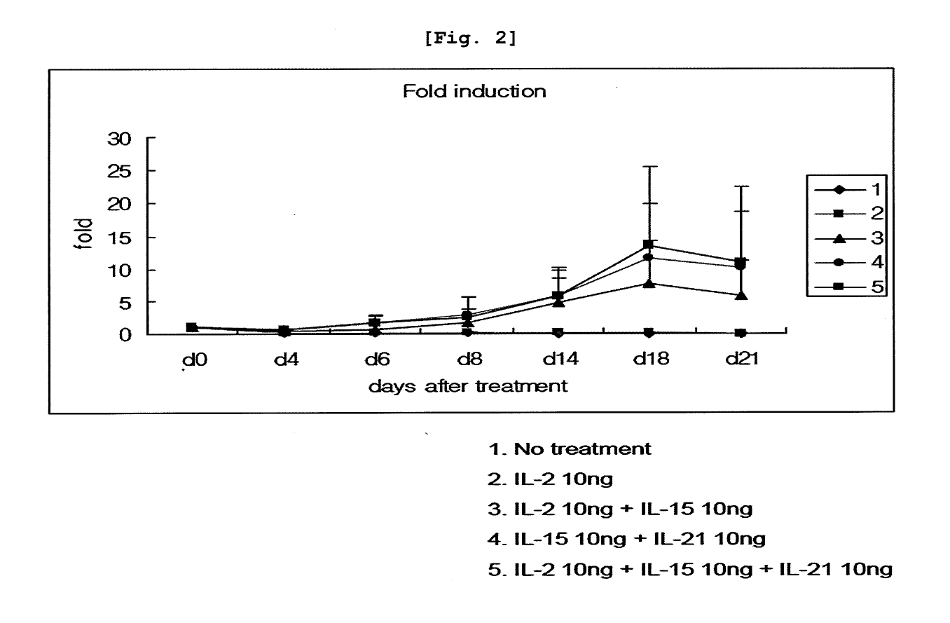

The effect of cytokine on the proliferation of NK cells was also investigated in this invention. To do so, cytokine non-treated CD3 negative cell group, IL-2 treated CD3 negative cell group, IL-2 and IL-15 co-treated CD3 negative cell group, IL-15 and IL-21 co-treated CD3 negative cell group, and IL-2, IL-15, and IL-21 co-treated CD3 negative cell group were cultured respectively. Then, the ratio of CD3⁻CD56⁺ NK cells was analyzed by using FACS. As a result, NK cell population was rather decreased in the group that was not treated with cytokine and in the group treated with IL-2 alone. In the meantime, the cell population was increased in the rest of the groups until the 18^{th} day. From the 18^{th} day, the cell population began to decrease. Among those groups, the cell group treated with all of IL-2, IL-15, and IL-21 and the group co-treated with IL-15 and IL-21 demonstrated good cell growth rates (see FIG. 2).

Therefore, it was confirmed that CD3 negative cell growth could be accelerated when IL-15 and IL-21 were co-treated or when all of IL-2, IL-15, and IL-21 were treated together.

In this invention, NK cell differentiation from hematopoietic stem cells was compared with that from CD3 negative cells. To do so, NK cell differentiation was induced from umbilical cord blood originated hematopoietic stem cells and CD3 negative cells respectively. Then, NK cell population rate was investigated by FACS. As a result, increased NK cell population with high purity was obtained in a short period of time from using CD3 negative cells rather than from using hematopoietic stem cells. Particularly, primary CD3 negative cells were obtained in a large scale, which could be proliferated further as many as 15 times. So, a large number of NK cells were obtained at last (see Table 3-Table 6).

The method to induce NK cell proliferation by using CD3 negative cells was proved to be more efficient to obtain a large amount of NK cells in a short period of time than the method to induce NK cell proliferation by using hematopoietic stem cells.

The present invention also provides a method for differentiating NK cells comprising the following steps:

1) preparing CD3 negative cells by eliminating CD3 positive T cells from umbilical cord blood derived mononuclear cells; and

2) culturing the CD3 negative cells after treating the cells of step 1) with cytokines.

In the above method, the preparation of CD3 negative cells in step 1) can be accomplished by either the method of using CD3 microbeads to give CD positive cells magnetism which would be filtered by MACS column to separate CD3 negative cells therefrom or the method of labeling CD3 negative T cells with fluorescence which would be separated by using Cell Sorter, but not always limited thereto.

In step 2) of the above method, it is preferred to treat two or more cytokines together which are selected from the group consisting of IL-2, IL-15, and IL-21. Particularly, co-treatment with IL-15 and IL-21 is preferred or co-treatment with all of IL-2, IL-15, and IL-21 is more preferred, but not always limited thereto.

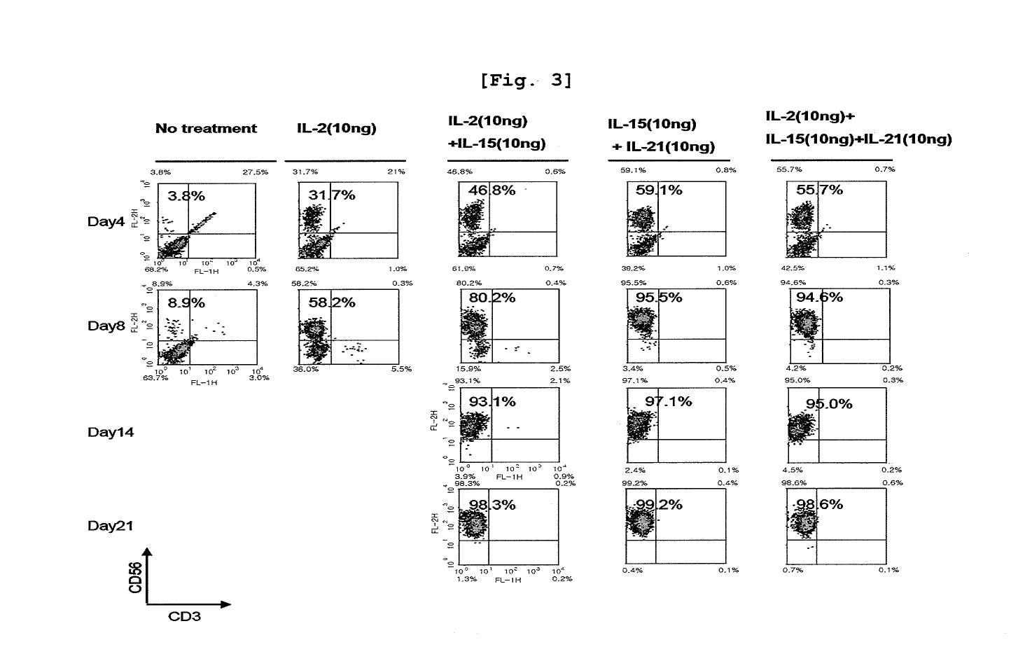

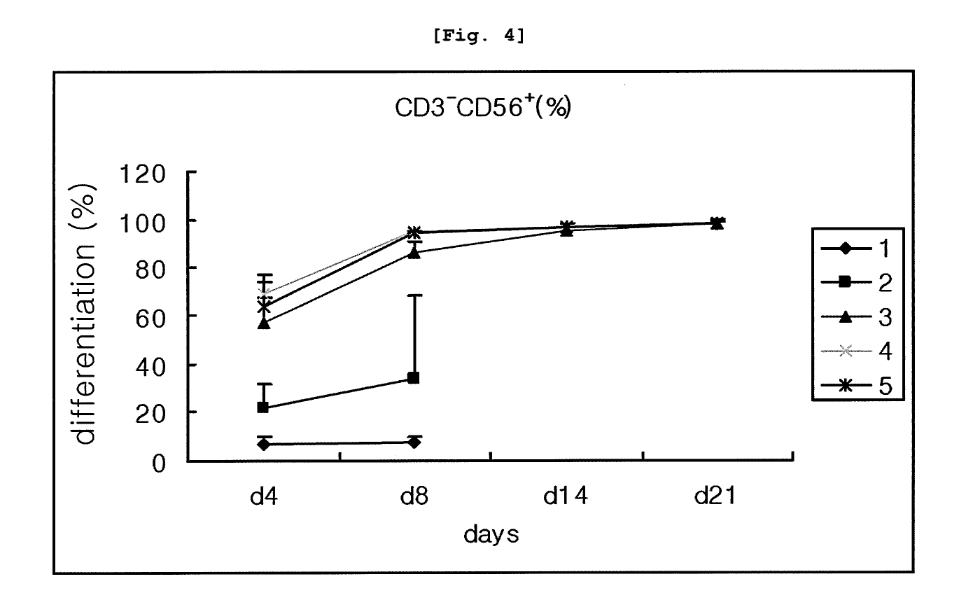

To investigate the effect of cytokine on NK cell differentiation, different combinations of cytokines were treated to CD3 negative cells in this invention, followed by analysis of NK cell population rate by using FACS. As a result, the group treated with two or more cytokines demonstrated high differentiation rate, compared with other groups treated with IL-2 alone or not treated with any cytokine. The cell group co-treated with two or more cytokines showed high differentiation rate reaching at least 90% since day 8. Those two groups each treated with two cytokines, IL-15 and IL-21, and treated with all three of them, IL-2, IL-15, and IL-21, showed similarly high differentiation rates (see FIG. 3 and FIG. 4).

Therefore, it was confirmed that co-treatment with IL-15 and IL-21 or co-treatment with IL-2, IL-15 and IL-21 was more efficient in inducing NK cell differentiation from CD3 negative cells.

In this invention, NK cell differentiation from hematopoietic stem cells was compared with that from CD3 negative cells. To do so, NK cell differentiation was induced from umbilical cord blood originated hematopoietic stem cells and CD3 negative cells respectively. Then, NK cell population rate was investigated by FACS. As a result, increased NK cell population with high purity was obtained in a short period of time from using CD3 negative cells rather than from using hematopoietic stem cells. Particularly, primary CD3 negative cells were obtained in a large scale, which could be proliferated further as many as 15 times. So, a large number of NK cells were obtained at last (see Table 3-Table 6).

The method to induce NK cell proliferation by using CD3 negative cells was proved to be more efficient to obtain a large amount of NK cells in a short period of time than the method to induce NK cell proliferation by using hematopoietic stem cells.

The present invention further provides a method for preparing NK cells with increased cytotoxicity comprising the following steps:

1) preparing CD3 negative cells by eliminating CD3 positive T cells from umbilical cord blood derived mononuclear cells; and

2) culturing the CD3 negative cells after treating the cells of step 1) with cytokines.

In the above method, the preparation of CD3 negative cells in step 1) can be accomplished by either the method of using CD3 microbeads to give CD positive cells magnetism which would be filtered by MACS column to separate CD3 negative cells therefrom or the method of labeling CD3 negative T cells with fluorescence which would be separated by using Cell Sorter, but not always limited thereto.

In step 2) of the above method, it is preferred to treat two or more cytokines together which are selected from the group consisting of IL-2, IL-15, and IL-21. Particularly, co-treatment with IL-15 and IL-21 is preferred or co-treatment with all of IL-2, IL-15, and IL-21 is more preferred, but not always limited thereto.

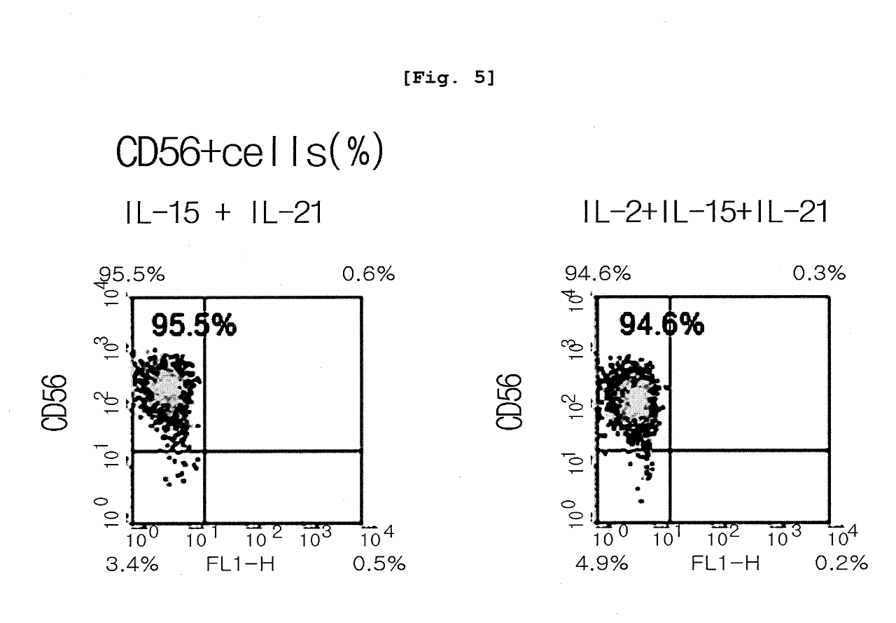

To investigate the effect of cytokine on NK cell activity, CD3 negative cells were treated with different combinations of cytokines, which would be differentiated into NK cells. Then, ⁵¹Cr secretion was investigated by using γ-counter and lactate dehydrogenase (LDH) activity was measured using a kit. As a result, higher cytotoxicity was observed in the group treated with at least two different cytokines than in the group treated with IL-21 alone or not treated with any cytokine. And, the group treated with IL-15 and IL-21 together showed comparatively high cytotoxicity, compared with the group treated with all of those three cytokines, IL-2, IL-15, and IL-21 (see FIG. 5-FIG. 7).

The above results suggest that co-treatment with IL-15 and IL-21 is more efficient in inducing differentiation of NK cells from CD3 negative cells and the resultant NK cells have higher cytotoxicity.

The present invention also provides a preventive and therapeutic composition for cancer containing the NK cells with increased cytotoxicity prepared by the said method.

The cancer herein is preferably selected from the group consisting of breast cancer, melanoma, stomach cancer, liver cancer, colon cancer, and lung cancer, but not always limited thereto.

The composition of the present invention can contain one or more active ingredients having the same or similar functions to the above NK cells.

The composition of the present invention can include one or more pharmaceutically acceptable carriers selected from the group consisting of saline, sterilized water, Ringer's solution, buffered saline, dextrose solution, maltodextrin solution, glycerol, ethanol, liposome and a mixture comprising one or more of those components. If necessary, a general additive such as an antioxidant and buffer can be additionally added. The composition of the present invention can be formulated in different forms including aqueous solutions, suspensions and emulsions for injection, pills, capsules, granules or tablets by mixing with diluents, dispersing agents, surfactants, binders and lubricants. The composition can further be prepared in suitable forms according to ingredients by the following method represented in Remington's Pharmaceutical Science (Mack Publishing Company, Easton Pa., 18th, 1990).

The composition of the present invention can be administered parenterally (for example, intravenous, hypodermic, peritoneal or local injection). The effective dosage of the composition can be determined according to weight, age, gender, health condition, diet, administration frequency, administration method, excretion and severity of a disease. The dosage is 0.0001˜500 mg/kg per day and preferably 0.01˜10 mg/kg per day, and administration frequency is once a day or preferably a few times a day.

The present inventors confirmed that the NK cells with increased cytotoxicity could be induced from CD3 negative cells and thus NK cells having anti-cancer cytotoxicity could be differentiated therefrom, suggesting that the cells of the invention can be effectively used for anti-cancer treatment.

The present invention also provides a method for treating cancer containing the step of administering a pharmaceutically effective dosage of the said composition to a subject having cancer.

The present invention also provides a method for preventing cancer containing the step of administering a pharmaceutically effective dosage of the said composition to a subject.

The cancer herein is preferably selected from the group consisting of breast cancer, melanoma, stomach cancer, liver cancer, colon cancer, and lung cancer, but not always limited thereto.

The composition of the present invention can contain one or more active ingredients having the same or similar functions to the above NK cells. The composition can also include, in addition to the above-mentioned effective ingredients, one or more pharmaceutically acceptable carriers for the administration.

The composition of the present invention can be administered parenterally (for example, intravenous, hypodermic, peritoneal or local injection). The effective dosage of the composition can be determined according to weight, age, gender, health condition, diet, administration frequency, administration method, excretion and severity of a disease. The dosage is 0.01˜5000 mg/kg per day and preferably 0.01˜10 mg/kg per day, and administration frequency is once a day or preferably a few times a day.

The present inventors confirmed that the NK cells with increased cytotoxicity could be induced from CD3 negative cells and thus NK cells having anti-cancer cytotoxicity could be differentiated therefrom, suggesting that the cells of the invention can be effectively used for anti-cancer treatment.

In addition, the present invention provides a use of the NK cells with increased cytotoxicity prepared by the method of the present invention for the production of a preventive and therapeutic composition for cancer.

The cancer herein is preferably selected from the group consisting of breast cancer, melanoma, stomach cancer, liver cancer, colon cancer, and lung cancer, but not always limited thereto.

The present inventors confirmed that the NK cells with increased cytotoxicity could be induced from CD3 negative cells and thus NK cells having anti-cancer cytotoxicity could be differentiated therefrom, suggesting that the cells of the invention can be effectively used for anti-cancer treatment.

Practical and presently preferred embodiments of the present invention are illustrative as shown in the following Examples, Experimental Examples and Manufacturing Examples.

However, it will be appreciated that those skilled in the art, on consideration of this disclosure, may make modifications and improvements within the spirit and scope of the present invention.

### Example 1

### Preparation of CD3 Negative Cells from Umbilical Cord Blood Originated Mononuclear Cells

Umbilical cord blood provided from hospitals (Department of Obstetrics & Gynecology, Konyang University Hospital and Department of Obstetrics & Gynecology, Chungnam National University Hospital, Korea, Each hospital IRB test was passed) for the purpose of research was diluted with RPMI 1640 at the ratio of 2:1. The prepared umbilical cord blood was carefully loaded on the upper part of Ficoll-Paque, followed by centrifugation at 20,000 rpm for 30 minutes to obtain mononuclear cell layer (MNC layer). Erythrocytes were eliminated from the cells obtained from the mononuclear cell layer to obtain mononuclear cells. Microbeads (Miltenyi Biotech) were added to the obtained mononuclear cells, followed by labeling. CD3 positive cells were eliminated by using CS column and Vario MACS to obtain CD3 negative cells. Particularly, CD3 microbeads (Miltenyi Biotech) recognized CD3ε chain so as to give magnetism to CD3 positive cells from mononuclear cells. Then, those microbead-attached CD3 positive cells in mononuclear cells were passed through MACS column that reacted with magnetism. As a result, CD3 positive cells remained in the column, while CD3 negative cells were passed through the column, by which the separation was completed. The purity of the obtained CD3 negative cells was investigated by flow cytometry (FACS). As a result, as shown in Table 1 and Table 2, the yield of CD3 negative cells from mononuclear cells was 32% (Table 1) and the purity of CD3 negative cells was 88% at average (Table 2 and FIG. 1).

TABLE 1

| | Experiment | Experiment | Experiment |
|---|---|---|---|
| | 1 | 2 | 3 |
| MNC | 28.7 × 10⁷ | 25 × 10⁷ | 25.2 × 10⁷ |
| CD3 negative cells | 7 × 10⁷ | 5.25 × 10⁷ | 7.7 × 10⁷ |
| Yield (CD34−CD3⁻/ | 24% | 21% | 30.5% |
| MNC) × 100 | | | |

| | |
|---|---|
| Average | 25.17% |

TABLE 2

| | CD3−CD56+ (%) | CD3−(CD3+) (%) | |
|---|---|---|---|
| Experiment 1 | 14.2 | 66.1 | (33.9) |
| Experiment 2 | 10.8 | 98.3 | (1.7) |
| Experiment 3 | 5.1 | 99.9 | (0.1) |
| Average | 10.03 | 88.1 | (11.9) |

### Example 2

### Effect of Cytokines on Proliferation of CD3 Negative Cells and Differentiation to NK Cells

CD3 negative cells isolated from umbilical cord blood were inoculated on 12-well plate (Falcon, USA) at the concentration of 1×10⁶ cells/ml. The cells were classified as the group non-treated with cytokine, the group treated with IL-2 alone, the group treated with IL-2 and IL-15 together, the group treated with IL-15 and IL-21 together, and the group treated with all of those three cytokines, IL-2, IL-15, and IL-21, followed by culture on Myelocult (Stem cell Technology) complete medium at 37□, 5% CO₂ incubator for 21 days. When the cell concentration reached 1×10⁶ cells/ml during the culture, the cells were distributed for sub-culture on another medium which had the same composition as the one primarily used. Cell number was counted on day 4, day 8, day 14, day 18, and day 21. The cells were conjugated with CD3 antibody and CD56 antibody on day 4, day 8, day 14, and day 21, followed by FACS to investigate CD3⁻CD56⁺ NK cell ratio.

As a result, cell population was not increased or rather reduced in the group not treated with cytokine and in the group treated with IL-2 alone. In the meantime, cell population was increased in the remaining three groups, however the population began to decrease from day 18. Only those groups co-treated with IL-2, IL-15, and IL-21 and co-treated with IL-15 and IL-21 showed relatively high cell growth similarly to each other (FIG. 2). Therefore, it was proved that the treatment of combinations of cytokines, particularly co-treatment of IL-15 and IL-21, could promote the proliferation of CD3 negative cells.

From the result of investigating NK cell ratio, it was confirmed that the group treated with at least two different cytokines exhibited higher differentiation rate, particularly 90% or up from day 8, than the group not-treated or treated with IL-2 alone. Among three combinations of cytokines, the combination of IL-2, IL-15, and IL-21, and the combination of IL-15 and IL-21 exhibited similarly higher differentiation rates (FIG. 3 and FIG. 4). In conclusion, the treatment of mixed cytokines, particularly the combination including IL-15 and IL-21, was proved to be efficient in inducing NK cell differentiation from CD3 negative cells.

### Example 3

### Effect of Cytokines on NK Cell Activity in CD3 Negative Cells

CD3 negative cells were treated with different combinations of cytokines as shown in Example 2 to induce differentiation to NK cells. Then, ⁵¹Cr secretion and lactate dehydrogenase (LDH) activity were investigated in those groups treated with the combination of IL-2, IL-15, and IL-21, and treated with the combination of IL-15 and IL-21, which demonstrated higher proliferation rate and differentiation rate (FIG. 5).

The differentiated NK cells were washed, followed by culture in 96 well round bottom plate loaded with the target cells, ⁵¹Cr-labeled K562 cells (10⁴/well), considering the ratio of effector:target, for 4 hours. Upon completion of the culture, 100 μl of culture supernatant was obtained, followed by the measurement of radioactivity by using γ-counter. And, the differentiated NK cells were washed, followed by culture in 96 well round bottom plate loaded with the target K562 cells, considering the ratio of effector:target, for 4 hours. 50 μl of culture supernatant was obtained, followed by reaction with LDH substrate at room temperature for 30 minutes. Then, LDH activity was measured.

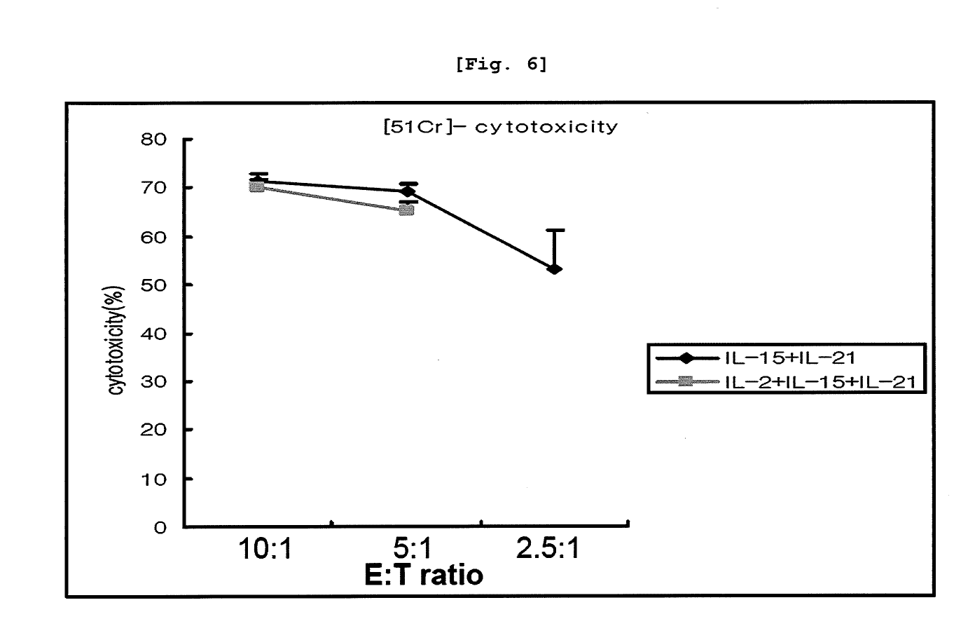

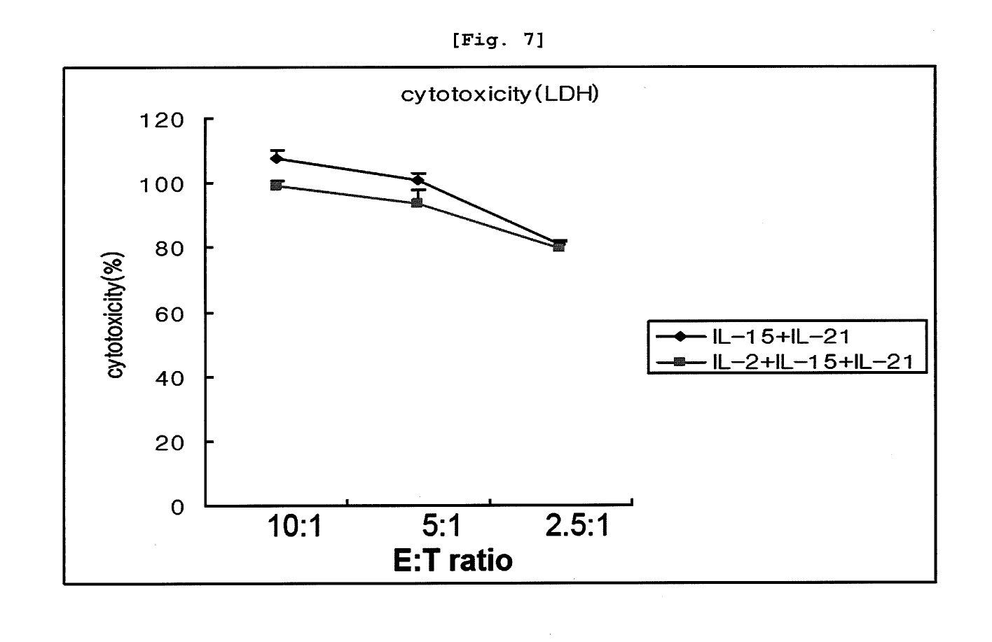

As a result, higher cytotoxicity was demonstrated in the group co-treated with IL-15 and IL-21 than in the group treated with all of those cytokines, IL-2, IL-15, and L-21 (FIG. 6 and FIG. 7). Therefore, it has been confirmed that the treatment of cytokine combination of IL-15 and IL-21 is more efficient in inducing NK cell differentiation from CD3 negative cells and those differentiated NK cells have higher cytotoxicity.

### Example 4

### Comparison of NK Cell Differentiation and Proliferation from CD3 Negative Cells and from Hematopoietic Stem Cells

<4-1> Isolation of Hematopoietic Stem Cells from Umbilical Cord Blood and Differentiation to NK Cells

Umbilical cord blood provided from hospitals for the purpose of research was diluted with RPMI 1640 at the ratio of 2:1. The prepared umbilical cord blood was carefully loaded on the upper part of Ficoll-Paque, followed by centrifugation at 20,000 rpm for 30 minutes to obtain MNC layer. Erythrocytes were eliminated from the cells obtained from the MNC layer to obtain mononuclear cells. Hematopoietic stem cell marker CD34 microbeads were added thereto for labeling, followed by the separation of CD34⁺ cells by using MS/RS column and MAC. The separated hematopoietic stem cells were inoculated in 12-well plate (Falcon, USA) at the concentration of 1×10⁶ cells/well, followed by culture in Myelocult (Stem Cell Technology) complete medium supplemented with human SCF (30 ng/ml, PeproTech, Rocky Hill, N.J.), human Flt3L (50 ng/ml, PeproTech, Rocky Hill, N.J.), human IL-(5 ng/ml, PeproTech), and hydrocortisone (10⁻⁶ M, stem cell Tech.) at 37□, 5% CO₂ incubator for 14 days. Three days later, half of the culture supernatant was replaced with the same fresh medium containing cytokines. For the differentiation to mature NK cells, HSCs were recovered 14 days later and then further cultured for additional 14 days in the presence of human IL-15 (30 ng/ml, PeproTech). Three days after the culture started, half of the medium was replaced with the same fresh medium containing cytokines.

<4-2> Comparison of NK Cell Proliferation and Differentiation from CD3 Negative Cells and from Hematopoietic Stem Cells

To compare NK cell differentiation and proliferation from hematopoietic stem cells and from CD3 negative cells, umbilical cord blood originated hematopoietic stem cells and CD3 negative cells were obtained. NK cell differentiation and proliferation were induced therefrom, followed by FACS.

As a result, as shown in Table 3-Table 5, the ratio of CD34⁺, the hematopoietic stem cells of umbilical cord blood was up to 1% (0.88% at average) (Table 3), indicating that initial cell population was too small (1-3×10⁶/50 ml of umbilical cord blood) to induce proliferation (average cell number, 1×10⁸ cells) (Table 4). Besides, differentiation to NK cells through precursors took 5 weeks. The resultant NK cells demonstrated even low purity of 60-90% (86% at average) (Table5) and big individual difference.

TABLE 3

| | Experiment | Experiment | Experiment | Experiment |
|---|---|---|---|---|
| | 1 | 2 | 3 | 4 |
| MNC | 2.8 × 10⁸ | 2.18 × 10⁸ | 2.8 × 10⁸ | 2.6 × 10⁸ |
| HSC | 1.4 × 10⁶ | 2.02 × 10⁶ | 3.5 × 10⁶ | 2.3 × 10⁶ |
| (CD34⁺ cell) | | | | |
| (CD34⁺/ | 0.5% | 0.92% | 1.25% | 0.88% |
| MNC) × 100 | | | | |

TABLE 4

| | Experiment | Experiment | Experiment | Experiment |
|---|---|---|---|---|
| | 1 | 2 | 3 | 4 |
| mNK (5 wk) | 1.13 × 10⁸ | 8.9 × 10⁸ | 1.38 × 10⁸ | 1.1 × 10⁸ |
| Fold | 80.7 | 44.0 | 39.4 | 54.7 |
| (mNK/HSC) | | | | |

TABLE 5

| | CD56⁺CD122⁺ (%) |
|---|---|
| Experiment 1 | 95.56 |
| Experiment 2 | 68.02 |
| Experiment 3 | 95.69 |
| Average | 86.42 |

In the meantime, as shown in Table 6, the method inducing differentiation from CD3 negative cells was confirmed to be able to yield NK cells with high purity in a large scale in a short period of time, compared with the method using hematopoietic stem cells (Table 6). That is, a large amount of initial CD3 negative cells could be obtained (5-8×10⁷/50 ml of umbilical cord blood), which would be proliferated up to 15 times more, so that NK cells would be obtained in a large scale (1×10⁹ cells at average). From the 8^{th} day of culture, NK cells with high purity of at least 95% (CD3⁻CD56⁺) could be obtained, suggesting that differentiation period was much shorter but purity was higher in this method, compared with the NK cells originated from haematopoietic stem cells.

TABLE 6

| | | Differen- | NK differen- | |
|---|---|---|---|---|
| | Initial | tiation | tiation | Final NK |
| | Cell No. | Period | Rate (%) | Cell No. |
| MNC | 2.6 × 10⁸ | — | — | — |
| HSC (CD34⁺ cell) | 2.3 × 10⁶ | 5 weeks | 86.4 | 1.1 × 10⁸ |
| CD3⁻ cell | 6.7 × 10⁷ | 8 days | 95.5 | 1.0 × 10⁹ |

Therefore, it was confirmed that the method of inducing NK cell differentiation from CD3 negative cells favored NK cell preparation in a large scale in a short period of time, compared with the method of inducing NK cells from haematopoietic stem cells.

### INDUSTRIAL APPLICABILITY

As explained hereinbefore, the present invention provides a more efficient and economic method for proliferating and differentiating NK cells, so that it can be used effectively for anti-cancer immune-cell therapy using NK cells which usually requires a huge number of NK cells.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

What is claimed is:

1. A method for proliferating NK cells comprising the following steps:
1) preparing CD3 negative cells by eliminating CD3 positive T cells from umbilical cord blood derived mononuclear cells; and
2) culturing the CD3 negative cells after treating the cells of step 1) with cytokines.

2. The method for proliferating NK cells according to claim 1, wherein the preparation of step 1) is performed by the following processes; making CD3 positive cells magnetic by using CD3 microbeads; and separating CD3 negative cells by using MACS column or separating CD3 negative cells by using cell sorter after labeling CD3 negative T cells with fluorescence.

3. The method for proliferating NK cells according to claim 1, wherein the treatment of cytokines of step 2) is performed with at least two cytokines together selected from the group consisting of IL-2, IL-15, and IL-21.

4. The method for proliferating NK cells according to claim 1, wherein the treatment of cytokines of step 2) is the co-treatment of IL-15 and IL-21.

5. A method for differentiating NK cells comprising the following steps:
1) preparing CD3 negative cells by eliminating CD3 positive T cells from umbilical cord blood derived mononuclear cells; and
2) culturing the CD3 negative cells after treating the cells of step 1) with cytokines.

6. The method for differentiating NK cells according to claim 5, wherein the preparation of step 1) is performed by the following processes; making CD3 positive cells magnetic by using CD3 microbeads; and separating CD3 negative cells by using MACS column or separating CD3 negative cells by using cell sorter after labeling CD3 negative T cells with fluorescence.

7. The method for differentiating NK cells according to claim 5, wherein the treatment of cytokines of step 2) is performed with at least two cytokines together selected from the group consisting of IL-2, IL-15, and IL-21.

8. The method for differentiating NK cells according to claim 5, wherein the treatment of cytokines of step 2) is the co-treatment of IL-15 and IL-21.

9. A method for preparing NK cells with increased cytotoxicity comprising the following steps:
1) preparing CD3 negative cells by eliminating CD3 positive T cells from umbilical cord blood derived mononuclear cells; and
2) culturing the CD3 negative cells after treating the cells of step 1) with cytokines.

10. The method for preparing NK cells with increased cytotoxicity according to claim 9, wherein the preparation of step 1) is performed by the following processes; making CD3 positive cells magnetic by using CD3 microbeads; and separating CD3 negative cells by using MACS column or separating CD3 negative cells by using cell sorter after labeling CD3 negative T cells with fluorescence.

11. The method for preparing NK cells with increased cytotoxicity according to claim 9, wherein the treatment of cytokines of step 2) is performed with at least two cytokines together selected from the group consisting of IL-2, IL-15, and IL-21.

12. The method for preparing NK cells with increased cytotoxicity according to claim 9, wherein the treatment of cytokines of step 2) is the co-treatment of IL-15 and IL-21.

13. A preventive and therapeutic composition for cancer containing the NK cells with increased cytotoxicity prepared by the method of claim 9.

14. The preventive and therapeutic composition for cancer according to claim 13, wherein the cancer is selected from the group consisting of breast cancer, melanoma, stomach cancer, liver cancer, colon cancer and lung cancer.

15. A method for treating cancer containing the step of administering a pharmaceutically effective dosage of the composition of claim 13 to a subject having cancer.

16. The method for treating cancer according to claim 15, wherein the cancer is selected from the group consisting of breast cancer, melanoma, stomach cancer, liver cancer, colon cancer and lung cancer.

17. A method for preventing cancer containing the step of administering a pharmaceutically effective dosage of the composition of claim 13 to a subject.

18. The method for preventing cancer according to claim 17, wherein the cancer is selected from the group consisting of breast cancer, melanoma, stomach cancer, liver cancer, colon cancer and lung cancer.

19. A use of the NK cells with increased cytotoxicity prepared by the method of claim 9 for the production of a preventive and therapeutic composition for cancer.

20. The use according to claim 19, wherein the cancer is selected from the group consisting of breast cancer, melanoma, stomach cancer, liver cancer, colon cancer and lung cancer.
